(19) United States
(12) Patent Application Publication
(10) Pub. No.: US 2025/0282960 A1
(43) Pub. Date: Sep. 11, 2025

(54) PAINT THINNER FORMULATION

(71) Applicant: HINDUSTAN PETROLEUM CORPORATION LIMITED, Bengaluru (IN)

(72) Inventors: Palanisamy Ravichandiran, Bengaluru (IN); Sandip Bhowmik, Bengaluru (IN); Sheshachala Srinivasa Narasimha, Bengaluru (IN)

(21) Appl. No.: 18/658,756

(22) Filed: May 8, 2024

(30) Foreign Application Priority Data
Mar. 7, 2024 (IN) 202441016307

(51) Int. Cl. C09D 7/20; C07D 231/50

(52) U.S. Cl. CPC C09D 7/20; C07D 231/50

(57) ABSTRACT

The present invention provides a paint thinner formulation that comprises an aliphatic hydrocarbon, an aromatic hydrocarbon, an alcohol and a solvency promotor. The paint thinner formulation of the present invention comprises an aliphatic hydrocarbon, an aromatic hydrocarbon, an alcohol and a solvency promotor comprising a compound of formula (3).

### CROSS-REFERENCE TO RELATED PATENT APPLICATIONS

The present application is a U.S. patent application claiming priority to Indian Application No. 202441016307, filed Mar. 7, 2024, the disclosure of which is incorporate herein by reference in its entirety for all purposes.

### FIELD OF THE INVENTION

The present invention relates to a paint thinner formulation. More particularly, the present invention relates to a paint thinner formulation comprising an aliphatic hydrocarbon, an aromatic hydrocarbon, an alcohol and a solvency promotor. The present invention also relates to a novel solvency promotor compound of formula (3) for use as a solvency promotor in the paint thinner formulation.

### BACKGROUND OF THE INVENTION

Mineral Turpentine Oil (MTO) is universally preferred and recommended as paint thinner or diluent by leading paint manufacturers for most general painting applications. In recent years, exponential growth in real estate and related infrastructure industries have spurred significant growth in the paints and thinners consumption. Alongside the growth, the paint industry has also diversified its product portfolio significantly, requiring more specialized thinners.

The patent CN111154319 talks about the paint thinner comprises (a) 3-15 weight % of emulsifier, (b) 18-30 weight % Et acetate, (c) 18-30 weight % of preservative, (d) 3-15 weight % of ethylene glycol monoethyl ether, (e) 5-10 weight % of calcium carbonate, (f) 3-8 weight % of ammonia, (g) 10-20 weight % of Bu acetate, (h) 1-5 weight % of butanol and, (i) 10-20 weight % of isophorone for High-efficiency paint thinner application.

The patent CN111154310 discloses about the preparation of environment-friendly and energy-saving paint thinner with strong solubility. The thinner comprises 2-10% of sunflower oil, 20-30% of Et acetate, 20-30% of acetone, 2-10% of ammonia, 5-10% of emulsifier, 3-8% of antioxidant, 10-20% of Bu acetate, 1-6% of glycerin, and 10-20% of calcium carbonate.

The patent WO2023010764 talks about the preparation and application of high-flash-point environment-friendly thinners. The presents invention relates to the tech. field of paint thinners and provides a high-flash-point environment-friendly thinner and a preparation method therefor and an application thereof. The paint thinner of the present invention is prepared from the following raw materials in parts by mass: 40-65 parts of tripropylene glycol ether, 20-30 parts of cyclohexanediol monomethyl ether, 10-20 parts of tetra-Bu orthosilicate, 5-25 parts of dipropylene glycol monomethyl ether, and 8-15 parts of acetic ester. The thinner is fresh and non-pungent in smell, healthy, safe, harmless, green, environment-friendly, good in thinning power, wide in a thinning range, capable of thinning a conventional paint in the field, fast in a film forming speed, and compact in a paint film.

Therefore, there lies a requirement for an improved paint thinner formulation that provide higher solvency and better surface adhesion of paint not only widens applicability but also reduces inconsistencies such as lumps, bubbling etc. Similarly, optimum drying time also significantly impacts the quality of paint application. Paints drying too fast can cause loss of adhesion and blistering. Slow drying on the other hand can cause loss of gloss and inconsistent paint thickness. Therefore, thinners with optimum viscosity and volatility are ideal for thinners. Another critical property for paint thinners is “Gloss”, a quantifiable parameter that defines the finish/paint quality.

### OBJECTIVES OF THE PRESENT INVENTION

The main objective of this invention is to provide a paint thinner formulation.

The second objective of the present invention is to provide a solar panel cleaning formulation that comprises an aliphatic hydrocarbon, an aromatic hydrocarbon, an alcohol and a solvency promotor.

The third objective of the present invention is to provide a paint thinner formulation that comprises an aliphatic hydrocarbon, an aromatic hydrocarbon, an alcohol and a solvency promotor comprising a novel compound (3).

The fourth objective of the present invention is to provide a novel solvency promotor compound (3).

The fifth objective of the present invention is to provide a process for preparation of novel solvency promotor compound (3).

The sixth objective of the present invention is to provide a paint thinner formulation with improved solvency, drying time, gloss and adhesion properties.

### SUMMARY OF THE PRESENT INVENTION

This summary is provided to introduce a selection of concepts, in a simplified format, that are further described in the detailed description of the invention. This summary is neither intended to identify key or essential inventive concepts of the invention nor is it intended to determine the scope of the invention.

In one of the embodiments, the present invention provides paint thinner formulation, comprising an aliphatic hydrocarbon, an aromatic hydrocarbon, an alcohol and a solvency promotor.

In one of the embodiments, the present invention provides paint thinner formulation, comprising an aliphatic hydrocarbon, an aromatic hydrocarbon, an alcohol and a solvency promotor wherein the aliphatic hydrocarbon is in a range of 87-89.8%, the aromatic hydrocarbon is in a range of 5-7%, the alcohol is in a range of 2-3% and the solvency promotor is in a range of 0.1-0.2%.

In one of the embodiments, the present invention provides paint thinner formulation, wherein the aliphatic hydrocarbon includes one or more selected from the group consisting of mineral turpentine oil (MTO), light Naphtha, heavy Naphtha, kerosene, light gas oil, heavy gas oil and a combination thereof.

In one of the preferred embodiments, the present invention provides paint thinner formulation, wherein the aliphatic hydrocarbon include one or more selected from the group consisting of mineral turpentine oil (MTO), light Naphtha, heavy Naphtha, kerosene and a combination thereof.

In one of the embodiments, the present invention provides paint thinner formulation, wherein the aromatic hydrocarbon include one or more selected from the group consisting of ortho-xylene, para-xylene, meta-xylene, mixed xylene, toluene, benzene, naphthalene, styrene, isopropylbenzene, methoxybenzene and a combination thereof.

In one of the preferred embodiments, the present invention provides paint thinner formulation, wherein the aromatic hydrocarbon includes one or more selected from the group consisting of ortho-xylene, para-xylene, meta-xylene, mixed xylene, toluene, benzene and a combination thereof.

In one of the embodiments, the present invention provides paint thinner formulation, wherein the alcohol includes one or more selected from the group consisting of 1-butanol, 1-pentanol, 1-propanol, ethanol, glycerol, isobutyl alcohol, methanol, isopentyl alcohol, isoamyl alcohol, secondary butyl alcohol, diacetone alcohol and a combination thereof.

In one of the preferred embodiments, the present invention provides paint thinner formulation, wherein the alcohol include one or more selected from the group consisting of 1-butanol, 1-propanol, ethanol, isobutyl alcohol, methanol, secondary butyl alcohol, diacetone alcohol and a combination thereof.

In one of the embodiments, the present invention provides paint thinner formulation, wherein the solvency promotor is selected from the group consisting of phenoxazines, phenothiazines, quinone-based heterocycles, furans, pyrroles, thiophenes, indoles, benzofurans, carbazoles, quinolines, isoquinolines, imidazoles, oxazoles, pyrazoles, pyridazines, pyrimidines, purine, antipyrines and a combination thereof.

In one of the embodiments, the present invention provides paint thinner formulation, wherein the solvency promotor is an antipyrines based solvency promotor.

In one of the embodiments, the present invention provides a paint thinner formulation, comprising mineral turpentine oil, xylene, diacetone alcohol and an antipyrines based solvency promotor.

In one of the embodiments, the present invention provides a paint thinner formulation, comprising mineral turpentine oil is in a range of 87-89.8%, xylene is in a range of 5-7%, diacetone alcohol is in a range of 2-3% and the antipyrines based solvency promotor is in a range of 0.1-0.2%.

In a preferred embodiment embodiments, the present invention provides a paint thinner formulation, wherein the solvency promotor is (4-(trifluoromethyl)-N-(2,3-dihydro-1,5-dimethyl-3-oxo-2-phenyl-1H-pyrazol-4-yl)-2-nitrobenzamide or compound of formula (3).

A compound of formula (3);

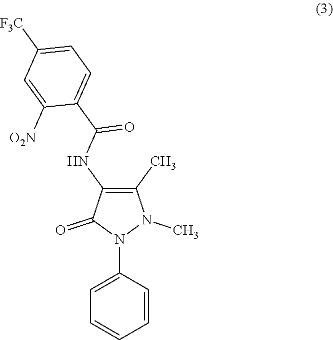 [SMILES: Cc1c(NC(=O)c2ccc(C(F)(F)F)cc2[N+](=O)[O-])c(=O)n(-c2ccccc2)n1C]

In yet one of the embodiments, the present invention provides a compound of formula (3), wherein the compound of formula (3) is (C₁₉H₁₅F₃N₄O₄).

In another embodiment, the present invention provides a process for preparation of compound of formula (3), the process comprising;
a) mixing 4-aminoantipyrine (1) and 2-nitro-4-(trifluoromethyl)benzoyl chloride (2) in dried acetone to obtain a reaction mixture;
b) refluxing the reaction mixture for 30 minutes to obtain a product mixture;
c) cooling the product mixture at room temperature;
d) precipitating the cooled product mixture by pouring the product mixture into ice—cold water to obtain a precipitate;
e) filtering the precipitate and washing the precipitate with 1000 ml of hot water;
f) drying the washed precipitate at 40° C. to obtain dried precipitate;
g) purifying the dried precipitate to obtain solvency promotor compound (3) 4-(trifluoromethyl)-N-(2,3-dihydro-1,5-dimethyl-3-oxo-2-phenyl-1H-pyrazol-4-yl)-2-nitrobenzamide).

### DETAILED DESCRIPTION OF THE INVENTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates. Unless otherwise defined, all technical and scientific terms used herein have the same meaning as commonly understood by one of ordinary skilled in the art to which this invention belongs. The system, methods, and examples provided herein are illustrative only and not intended to be limiting.

Before the present process and methods are described, it is to be understood that this invention is not limited to compounds, formulas, or steps described, as such may, of course, vary. It is also to be understood that the terminology used herein is to describe particular embodiments only and is not intended to be limiting.

Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context dictates otherwise. Thus, for example, reference to “a compound” includes a plurality of such compounds, and reference to “the step” includes reference to one or more steps and equivalents thereof known to those skilled in the art, and so forth.

The term “some” as used herein is defined as “none, or one, or more than one, or all.” Accordingly, the terms “none,” “one,” “more than one,” “more than one, but not all” or “all” would all fall under the definition of “some.” The term “some embodiments” may refer to no embodiments or to one embodiment or to several embodiments or to all embodiments. Accordingly, the term “some embodiments” is defined as meaning “no embodiment, or one embodiment, or more than one embodiment, or all embodiments.”

The terminology and structure employed herein is for describing, teaching and illuminating some embodiments and their specific features and elements and does not limit, restrict or reduce the spirit and scope of the claims or their equivalents.

More specifically, any terms used herein such as but not limited to “includes,” “comprises,” “has,” “consists,” and grammatical variants thereof do NOT specify an exact limitation or restriction and certainly do NOT exclude the possible addition of one or more features or elements, unless otherwise stated, and furthermore must NOT be taken to exclude the possible removal of one or more of the listed features and elements, unless otherwise stated with the limiting language “MUST comprise” or “NEEDS TO include.”

Whether or not a certain feature or element was limited to being used only once, either way it may still be referred to as “one or more features” or “one or more elements” or “at least one feature” or “at least one element.” Furthermore, the use of the terms “one or more” or “at least one” feature or element do NOT preclude there being none of that feature or element, unless otherwise specified by limiting language such as “there NEEDS to be one or more . . . ” or “one or more element is REQUIRED.”

Unless otherwise defined, all terms, and especially any technical and/or scientific terms, used herein may be taken to have the same meaning as commonly understood by one having an ordinary skill in the art.

Reference is made herein to some “embodiments.” It should be understood that an embodiment is an example of a possible implementation of any features and/or elements presented in the attached claims. Some embodiments have been described for the purpose of illuminating one or more of the potential ways in which the specific features and/or elements of the attached claims fulfil the requirements of uniqueness, utility and non-obviousness.

Use of the phrases and/or terms such as but not limited to “a first embodiment,” “a further embodiment,” “an alternate embodiment,” “one embodiment,” “an embodiment,” “multiple embodiments,” “some embodiments,” “other embodiments,” “further embodiment”, “furthermore embodiment”, “additional embodiment” or variants thereof do NOT necessarily refer to the same embodiments. Unless otherwise specified, one or more particular features and/or elements described in connection with one or more embodiments may be found in one embodiment, or may be found in more than one embodiment, or may be found in all embodiments, or may be found in no embodiments. Although one or more features and/or elements may be described herein in the context of only a single embodiment, or alternatively in the context of more than one embodiment, or further alternatively in the context of all embodiments, the features and/or elements may instead be provided separately or in any appropriate combination or not at all. Conversely, any features and/or elements described in the context of separate embodiments may alternatively be realized as existing together in the context of a single embodiment.

Any particular and all details set forth herein are used in the context of some embodiments and therefore should NOT be necessarily taken as limiting factors to the attached claims. The attached claims and their legal equivalents can be realized in the context of embodiments other than the ones used as illustrative examples in the description below.

Embodiments of the present invention will be described below in detail with reference to the accompanying drawings.

The present invention is drawn to a paint thinner formulation, comprising: an aliphatic hydrocarbons, an aromatic hydrocarbons, an alcohols and a solvency promotor. The paint thinner formulation of the present invention shows higher solvency and better surface adhesion of paint improved solvency, optimum drying time as paints drying too fast can cause loss of adhesion and blistering, improved gloss and adhesion properties that not only widens applicability but also reduces inconsistencies such as lumps, bubbling etc. The present invention is drawn to a paint thinner formulation that comprises an aliphatic hydrocarbon, an aromatic hydrocarbon, an alcohol and a solvency promotor comprising a novel compound (3).

The present invention is also drawn to a novel solvency promotor compound (3).

The present invention also relates to a process for preparation of novel solvency promotor compound (3).

The present invention is also drawn to a paint thinner formulation for general paint applications with overall superior finish (gloss) and performance. Solvency was envisioned as one of the core properties that influences drying time, gloss and overall finish of the paint.

In one of the embodiments, the present invention provides paint thinner formulation, comprising an aliphatic hydrocarbon, an aromatic hydrocarbon, an alcohol and a solvency promotor.

In one of the embodiments, the present invention provides paint thinner formulation, comprising an aliphatic hydrocarbon, an aromatic hydrocarbon, an alcohol and a solvency promotor wherein the aliphatic hydrocarbon is in a range of 87-89.8%, the aromatic hydrocarbon is in a range of 5-7%, the alcohol is in a range of 2-3% and the solvency promotor is in a range of 0.1-0.2%.

In one of the embodiments, the present invention provides paint thinner formulation, wherein the aliphatic hydrocarbon includes one or more selected from the group consisting of mineral turpentine oil (MTO), light Naphtha, heavy Naphtha, kerosene, light gas oil, heavy gas oil and a combination thereof.

In one of the preferred embodiments, the present invention provides paint thinner formulation, wherein the aliphatic hydrocarbon includes one or more selected from the group consisting of mineral turpentine oil (MTO), light Naphtha, heavy Naphtha, kerosene and a combination thereof.

In one of the embodiments, the present invention provides paint thinner formulation, wherein the aromatic hydrocarbon includes one or more selected from the group consisting of ortho-xylene, para-xylene, meta-xylene, mixed xylene, toluene, benzene, naphthalene, styrene, isopropylbenzene, methoxybenzene and a combination thereof.

In one of the preferred embodiments, the present invention provides paint thinner formulation, wherein the aromatic hydrocarbon includes one or more selected from the group consisting of ortho-xylene, para-xylene, meta-xylene, mixed xylene, toluene, benzene and a combination thereof.

In one of the embodiments, the present invention provides paint thinner formulation, wherein the alcohol includes one or more selected from the group consisting of 1-butanol, 1-pentanol, 1-propanol, ethanol, glycerol, isobutyl alcohol, methanol, isopentyl alcohol, isoamyl alcohol, secondary butyl alcohol, diacetone alcohol and a combination thereof.

In one of the preferred embodiments, the present invention provides paint thinner formulation, wherein the alcohol include one or more selected from the group consisting of 1-butanol, 1-propanol, ethanol, isobutyl alcohol, methanol, secondary butyl alcohol, diacetone alcohol and a combination thereof.

In one of the embodiments, the present invention provides paint thinner formulation, wherein the solvency promotor is selected from the group consisting of phenoxazines, phenothiazines, quinone-based heterocycles, furans, pyrroles, thiophenes, indoles, benzofurans, carbazoles, quinolines, isoquinolines, imidazoles, oxazoles, pyrazoles, pyridazines, pyrimidines, purine, antipyrines and a combination thereof.

In one of the embodiments, the present invention provides paint thinner formulation, wherein the solvency promotor is an antipyrines based solvency promotor.

In one of the embodiments, the present invention provides a paint thinner formulation, comprising mineral turpentine oil, xylene, diacetone alcohol and an antipyrines based solvency promotor.

In one of the embodiments, the present invention provides a paint thinner formulation, comprising mineral turpentine oil is in a range of 87-89.8%, xylene is in a range of 5-7%, diacetone alcohol is in a range of 2-3% and the antipyrines based solvency promotor is in a range of 0.1-0.2%.

In a preferred embodiment embodiments, the present invention provides a paint thinner formulation, wherein the solvency promotor is (4-(trifluoromethyl)-N-(2,3-dihydro-1,5-dimethyl-3-oxo-2-phenyl-1H-pyrazol-4-yl)-2-nitrobenzamide or compound of formula (3).

A compound of formula (3);

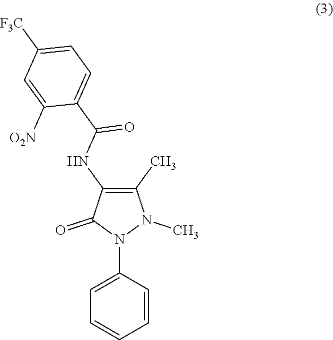 [SMILES: Cc1c(NC(=O)c2ccc(C(F)(F)F)cc2[N+](=O)[O-])c(=O)n(-c2ccccc2)n1C]

In yet one of the embodiments, the present invention provides a compound of formula (3), wherein the compound of formula (3) is (C₁₉H₁₅F₃N₄O₄).

In another embodiment, the present invention provides a process for preparation of compound of formula (3), the process comprising;
a) mixing 4-aminoantipyrine (1) and 2-nitro-4-(trifluoromethyl)benzoyl chloride (2) in dried acetone to obtain a reaction mixture;
b) refluxing the reaction mixture for 30 minutes to obtain a product mixture;
c) cooling the product mixture at room temperature;
d) precipitating the cooled product mixture by pouring the product mixture into ice—cold water to obtain a precipitate;
e) filtering the precipitate and washing the precipitate with 1000 ml of hot water;
f) drying the washed precipitate at 40° C. to obtain dried precipitate;
g) purifying the dried precipitate to obtain solvency promotor compound (3) 4-(trifluoromethyl)-N-(2,3-dihydro-1,5-dimethyl-3-oxo-2-phenyl-1H-pyrazol-4-yl)-2-nitrobenzamide).

MTO based paint thinner formulations, also referred to as thinners or solvents or formulation/s were prepared, and their solvency was evaluated through UV-Visible spectroscopy.

Further studies were carried out through Sigma Test & Research Center (NABL accredited lab) to evaluate superiority of the paint thinner formulations in terms of gloss, drying time and surface adhesion and conformity with all other specifications as per IS: 2932:2003 & IS:101 (BIS standard for paints).

### Methodology

### Preparation of Solvency Promotor Compound of Formula (3)

The starting materials such as 4-aminoantipyrine (2.03 g, 10 mmol) and 2-nitro-4-(trifluoromethyl)benzoyl chloride (2.53 g, 10 mmol) was mixed thoroughly in a dried acetone and the reaction mixture was refluxed for 30 min. After the completion of the reaction, the product mixture was cooled to room temperature and the crude mixture was poured into the ice-cold water. The formed pale-yellow precipitate was filtered, thoroughly washed with hot water (1000 mL) and dried at 40° C. The crude product was purified by column chromatography (silica gel 100-200 mesh, ethyl acetate:hexane, 1:1) to afford compound 4-(trifluoromethyl)-N-(2,3-dihydro-1,5-dimethyl-3-oxo-2-phenyl-1H-pyrazol-4-yl)-2-nitrobenzamide (3) as pale-yellow crystals (75%).

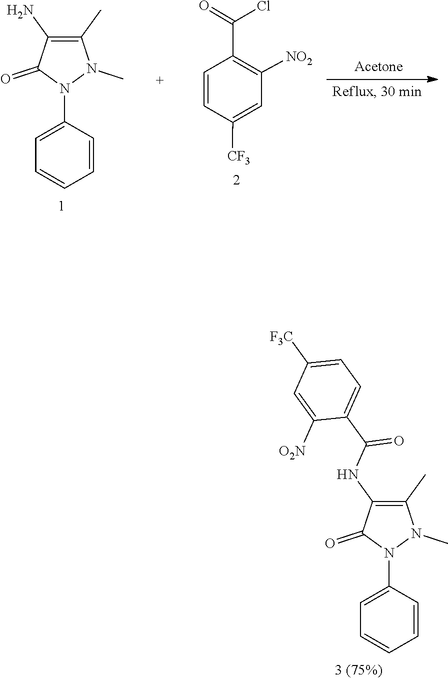

### 1.1. Preparation and Analysis of MTO Blends

Initially, the formulations blends with plain MTO were made. The organic component xylene, diacetone alcohol, and the solvency promotor (4-(trifluoromethyl)-N-(2,3-dihydro-1,5-dimethyl-3-oxo-2-phenyl-1H-pyrazol-4-yl)-2-nitrobenzamide, compound 3) was used as blending components. The composition of the various blends is given in the Table 1. It is noteworthy to mention, the solvent systems or paint thinner formulations without solvency promotor were carried out and it was found that formulation F-3 is a superior solvent system to improve the solvency of paints. Moreover, the different concentrations of compound (3) was studied with F-3, the concentration 0.2% was optimized concentration with improved solvency. Thus, the concentration, 0.2% was used in further paint thinner formulations.

TABLE 1
The complete details of finalized paint thinner formulations of
MTO with different chemical compositions are tabulated below.

| | | | | | Solvency |
|---|---|---|---|---|---|
| | | MTO (%, | | Diacetone | promotor |
| S. | | volume/ | Xylene | alcohol | (C₁₉H₁₅F₃N₄O₄) |
| No | Formulations | volume) | (%, v/v) | (%, v/v) | (%, v/v) |
| 1. | Blank | 99.8 | 0 | 0 | 0.2 |
| 2. | F-1 | 94.8 | 2.5 | 2.5 | 0.2 |
| 3. | F-2 | 90 | 7 | 3 | 0 |
| 4. | F-3 | 89.8 | 7 | 3 | 0.2 |
| 5. | F-4 | 79.8 | 10 | 10 | 0.2 |
| 6. | F-5 | 59.8 | 20 | 20 | 0.2 |
| 7. | F-6 | 69.8 | 15 | 15 | 0.2 |
| 8. | F-7 | 74.8 | 12.5 | 12.5 | 0.2 |
| 9. | F-8 | 89.8 | 5 | 5 | 0.2 |
| 10. | F-9 | 93.8 | 3 | 3 | 0.2 |
| 11. | Benchmark | 100 | 0 | 0 | 0 |

### Sample Preparation for UV-Vis Analysis.

100 mg of each type of paint was placed in a 10 mL glass vial. 5 mL of each from different paint thinner formulations were added slowly to the paint without agitating it for 2 hours. About, 3 mL of the supernatant solvent was then filtered through nylon membrane (0.2 μM) and UV-Vis's spectrum was recorded.

### Performance: Gloss, Drying Time, Abrasion and Adhesion

Performance evaluation was carried out by Sigma Test lab (NABL accredited) in accordance with requisite paint specifications of IS: 2932:2003 & IS:101.

### Results and Discussion

### Solvency: UV-Visible Spectroscopy

Considering complete visible wavelength range (400-800 nm), color pigments of yellow (complimentary λ=420-450 nm), red (complimentary λ=480-520 nm) and Blue (complimentary λ=700-750 nm) were selected to maximize coverage. It is to be noted, as the chosen colors are the elementary colors, most of the other conceivable color combinations can be prepared by proportionally blending two or more of these three colors.

The following paints from leading manufacturers were selected for this purpose: 1) Asian Paints Premium Enamel (Yellow), Asian Paints Premium Enamel (Red); 2) Asian Paints Premium Enamel (Blue). The results are given in the Table 2.

TABLE 2
UV-Vis data of different paints analyzed and their respective absorption intensity.

| Paint thinner | Red | | Yellow | | Blue | | Flash |
|---|---|---|---|---|---|---|---|
| formulations | λₘₐₓ (nm) | Abs | λₘₐₓ (nm) | Abs | λₘₐₓ (nm) | Abs | Point (° C.) |
| Blank | 505 | 0.2 | 423 | 0.2 | 725 | 0.1 | 38 |
| F-1 | 505 | 0.3 | 423 | 0.2 | 725 | 0.2 | 32.5 |
| F-2 | 505 | 0.5 | 423 | 0.4 | 725 | 0.2 | 35 |
| F-3 | 505 | 0.6 | 423 | 0.55 | 725 | 0.5 | 34.5 |
| F-4 | 505 | 0.5 | 423 | 0.2 | 725 | 0 | 31.5 |
| F-5 | 505 | 0.5 | 423 | 0.4 | 725 | 0 | 19 |
| F-6 | 505 | 0.3 | 423 | 0.3 | 725 | 0 | 11 |
| F-7 | 505 | 0.1 | 423 | 0.3 | 725 | 0 | 4.5 |
| F-8 | 505 | 0.1 | 423 | 0.4 | 725 | 0.3 | 4 |
| F-9 | 505 | 0.2 | 423 | 0.2 | 725 | 0.4 | 5 |
| Benchmark | 505 | 0.4 | 423 | 0.3 | 725 | 0.4 | 32.5 |

Based on the UV-Vis result, the formulation F-3 is showing the superior performance with better flash point. In this regard, the formulation 3 was selection for further solvency and gloss improving studies.

Performance: Gloss, Drying time, Abrasion and Adhesion without the Solvency Promotor

Performance screening was completed on red, yellow and blue paint by Sigma Test labs. For all tests 3:1 (V/V) paint to thinner formulation ratio was used. Results are shown in Tables 3-5.

TABLE 3
Performance evaluation of paint thinner formulation (F-3) with red premium enamel paint.

| | | | | Formulation | Formulation |
|---|---|---|---|---|---|
| | | | | without | with solvency |
| | | | | solvency | promotor |
| S. | | Requirement As Per IS: | Base MTO | promotor | (C₁₉H₁₅F₃N₄O₄) |
| No. | Test Parameters | 2932: 2003 & IS: 101 | (Red) | (Red) | (Red) |
| 1. | Water Resistance | — | Passes | Passes | Passes |
| | | | the test | the test | the test |
| 2. | Drying Time, Hrs | — | — | — | — |
| | Surface Dry | 4 h Max | 4 | 4 | 3.5 |
| | Hard Dry | 12 h Max | 12 | 12 | 11.5 |
| 3. | Color | Close match to the | Red | Red | Red |
| | | specified IS colour or | | | |
| | | to an agreed colour | | | |
| 4. | Gloss (60°) | Above 71 | 76.6 | 78.4 | 86.0 |
| | Bend Test | No visual cracks or | Passes | Passes | Passes |
| | | detachment of coating. | the test | the test | the test |
| | Loss in coating, micron | — | 28.275 | 19.15 | 4.9 |
| | Loss in weight, g | — | 0.0416 | 0.0402 | 0.0366 |
| 5. | Resistance to Alkali | Shall not show signs of | Passes | Passes | Passes |
| | | disintegration. No color | the test | the test | the test |
| | | change | | | |
| 6. | Pencil Hardness test | — | 2H | 3H | 4H |
| 7. | Temperature Stability | Paint shall not gel or | Passes | Passes | Passes |
| | | increase in viscosity more | the test | the test | the test |
| | | than 20% of the original | | | |
| | | value. For finishing paint, | | | |
| | | change in gloss value shall | | | |
| | | not be more than 5 units | | | |
| | | from that of original value. | | | |
| 8. | Protection against | No corrosion shall find | Passes | Passes | Passes |
| | Corrosion (168 hrs) | | the test | the test | the test |
| 9. | Flash Point ° C. | Not below 30° C. | 54 | 52 | 50 |

TABLE 4
Performance evaluation of paint thinner formulation (F-3) with yellow premium enamel paint.

| | | | | Formulation | Formulation |
|---|---|---|---|---|---|
| | | | | without | with solvency |
| | | | | solvency | promotor |
| S. | | Requirement As Per IS: | Base MTO | promotor | (C₁₉H₁₅F₃N₄O₄) |
| No. | Test Parameters | 2932: 2003 & IS: 101 | (Yellow) | (Yellow) | (Yellow) |
| 1. | Water Resistance | — | Passes | Passes | Passes |
| | | | the test | the test | the test |
| 2. | Drying Time, Hrs | — | — | — | — |
| | Surface Dry | 4 h Max | 4 | 4 | 3 |
| | Hard Dry | 12 h Max | 12 | 12 | 11 |
| 3. | Color | Close match to the | Yellow | Yellow | Yellow |
| | | specified IS colour or | | | |
| | | to an agreed colour | | | |
| 4. | Gloss (60°) | Above 71 | 73.2 | 77.4 | 84.2 |
| | Bend Test | No visual cracks or | Passes | Passes | Passes |
| | | detachment of coating. | the test | the test | the test |
| | Loss in coating, micron | — | 31.012 | 23.02 | 13.4 |
| | Loss in weight, g | — | 0.0712 | 0.0609 | 0.0482 |
| 5. | Resistance to Alkali | Shall not show signs of | Passes | Passes | Passes |
| | | disintegration. No color | the test | the test | the test |
| | | change | | | |
| 6. | Pencil Hardness test | — | 2H | 3H | 4H |
| 7. | Temperature Stability | Paint shall not gel or | Passes | Passes | Passes |
| | | increase in viscosity more | the test | the test | the test |
| | | than 20% of the original | | | |
| | | value. For finishing paint, | | | |
| | | change in gloss value shall | | | |
| | | not be more than 5 units | | | |
| | | from that of original value. | | | |
| 8. | Protection against | No corrosion shall find | Passes | Passes | Passes |
| | Corrosion (168 hrs) | | the test | the test | the test |
| 9. | Flash Point ° C. | Not below 30° C. | 54 | 52 | 50 |

TABLE 5
Performance evaluation of paint thinner formulation (F-3) with blue premium enamel paint.

| | | | | | Formulation |
|---|---|---|---|---|---|
| | | | | Formulation | with solvency |
| | | | | without | promotor |
| S. | | Requirement As Per IS: | Base MTO | promotor | (C₁₉H₁₅F₃N₄O₄) |
| No. | Test Parameters | 2932: 2003 & IS: 101 | (Blue) | (Blue) | (Blue) |
| | Water Resistance | — | Passes | Passes | Passes |
| | | | the test | the test | the test |
| 2. | Drying Time, Hrs | — | — | — | — |
| | Surface Dry | 4 h Max | 4 | 4 | 3.5 |
| | Hard Dry | 12 h Max | 12 | 12 | 11 |
| 3. | Color | Close match to the | Blue | Blue | Blue |
| | | specified IS colour or | | | |
| | | to an agreed colour | | | |
| 4. | Gloss (60°) | Above 71 | 67.2 | 70.3 | 78.0 |
| | Bend Test | No visual cracks or | Passes | Passes | Passes |
| | | detachment of coating. | the test | the test | the test |
| | Loss in coating, micron | — | 27.589 | 20.12 | 8.9 |
| | Loss in weight, g | — | 0.219 | 0.178 | 0.0748 |
| 5. | Resistance to Alkali | Shall not show signs of | Passes | Passes | Passes |
| | | disintegration. No color | the test | the test | the test |
| | | change | | | |
| 6. | Pencil Hardness test | — | 2H | 3H | 4H |
| 7. | Temperature Stability | Paint shall not gel or | Passes | Passes | Passes |
| | | increase in viscosity more | the test | the test | the test |
| | | than 20% of the original | | | |
| | | value. For finishing paint, | | | |
| | | change in gloss value shall | | | |
| | | not be more than 5 units | | | |
| | | from that of original value. | | | |
| 8. | Protection against | No corrosion shall find | Passes | Passes | Passes |
| | Corrosion (168 hrs) | | the test | the test | the test |
| 9. | Flash Point ° C. | Not below 30° C. | 54 | 52 | 50 |

As can be seen from the results, paint thinner formulation (F-3) has shown significantly improved gloss (more than 12.2% for red, 15% for yellow and 16% for blue paints compared to base MTO), reduced drying time and improved abrasion resistance in terms of loss in coating thickness. The performance in terms of adhesion and hardness was also found to be comparable to base MTO.

### Conclusions

A MTO based blend was developed for premium paint thinner applications. The thinner was tested on a wide variety of paints and was certified by an external agency to show highly improved gloss (12.2% for red, 15% for yellow and 16% for blue paints), superior abrasion resistance and reduced drying time. The paint thinner formulation was also tested and certified by external, NABL accredited lab to show compliance with complete standard specifications as per IS 2932:2003 & IS: 101. Moreover, the solvency promotor (4-(trifluoromethyl)-N-(2,3-dihydro-1,5-dimethyl-3-oxo-2-phenyl-1H-pyrazol-4-yl)-2-nitrobenzamide, compound 3) (50 M or 0.2%) improved the solvency, gloss and drying time.

Calculation to find the percentage of gloss efficiency.

| | | | Gloss | | | |
|---|---|---|---|---|---|---|
| | | | Formulation | | | |
| | | Gloss | with solvency | | | % |
| | | Base | promotor | | | Improve- |
| S. | | MTO | (C₁₉H₁₅F₃N₄O₄) | Gloss | | ment in |
| No. | Paint | (Blue) | (Blue) | Difference | Calculation | Gloss |
| 1. | Red | 76.6 | 86.0 | 9.4 | $\begin{matrix}Percentage of increase = \frac{\vert 76.6 - 86 \vert}{76.6} \\ = \frac{9.4}{76.6} \\ = 0.12271540469974 \\ = 12.27 \%\end{matrix}$ | 12.2% |
| 2. | Yellow | 73.2 | 84.2 | 11 | $\begin{matrix}Percentage of increase = \frac{\vert 73.2 - 84.2 \vert}{73.2} \\ = \frac{11}{73.2} \\ = 0.15027322404372 \\ = 15.02 \%\end{matrix}$ | 15% |
| 3. | Blue | 67.2 | 78.0 | 10.8 | $\begin{matrix}Percentage of increase \frac{\vert 67.2 - 78 \vert}{67.2} \\ = \frac{10.8}{67.2} \\ = 0.16071428571429 \\ = 16. 7 \%\end{matrix}$ | 16% |

### Technical Advantages of the Invention

The present invention has the following advantage over the prior arts:
The paint thinner formulation of the present invention shows highly improved gloss (12.2% for red, 15% for yellow and 16% for blue paints).
The current formulation is superior to reduce the consumption of paint thinner to dilute the paint.
The current formulation significantly reduced the drying time than the base solvent MTO).
The current formulation possesses the superior abrasion resistance towards the paint.

1. A paint thinner formulation, comprising: an aliphatic hydrocarbon, an aromatic hydrocarbon, an alcohol and a solvency promotor.

2. The paint thinner formulation as claimed in claim 1, wherein the aliphatic hydrocarbon is in a range of 87-89.8%, the aromatic hydrocarbon is in a range of 5-7%, the alcohol is in a range of 2-3% and the solvency promotor is in a range of 0.1-0.2%.

3. The paint thinner formulation as claimed in claim 1, wherein the aliphatic hydrocarbon include one or more selected from the group consisting of mineral turpentine oil, light Naphtha, heavy Naphtha, kerosene, light gas oil, heavy gas oil and a combination thereof.

4. The paint thinner formulation as claimed in claim 1, wherein the aromatic hydrocarbon include one or more selected from the group consisting of ortho-xylene, para-xylene, meta-xylene, mixed xylene, toluene, benzene, naphthalene, styrene, isopropylbenzene, methoxybenzene and a combination thereof.

5. The paint thinner formulation as claimed in claim 1, wherein the alcohol include one or more selected from the group consisting of 1-butanol, 1-pentanol, 1-propanol, ethanol, glycerol, isobutyl alcohol, methanol, isopentyl alcohol, isoamyl alcohol, secondary butyl alcohol, diacetone alcohol and a combination thereof.

6. The paint thinner formulation as claimed in claim 1, wherein the solvency promotor is selected from the group consisting of phenoxazines, phenothiazines, quinone-based heterocycles, furans, pyrroles, thiophenes, indoles, benzofurans, carbazoles, quinolines, isoquinolines, imidazoles, oxazoles, pyrazoles, pyridazines, pyrimidines, purine, antipyrines and a combination thereof.

7. A paint thinner formulation comprising: mineral turpentine oil, xylene, diacetone alcohol and an antipyrines based solvency promotor.

8. The pain thinner formulation as claimed in claim 7, wherein the solvency promotor is (4-(trifluoromethyl)-N-(2,3-dihydro-1,5-dimethyl-3-oxo-2-phenyl-1H-pyrazol-4-yl)-2-nitrobenzamide.

9. A compound of formula (3)

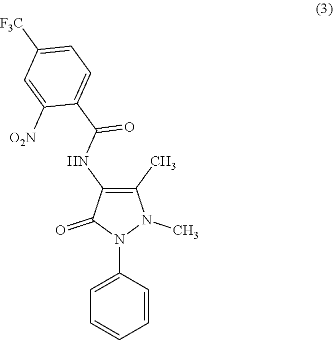 [SMILES: Cc1c(NC(=O)c2ccc(C(F)(F)F)cc2[N+](=O)[O-])c(=O)n(-c2ccccc2)n1C]

10. A process for producing the compound of claim 9, the process comprising:
a) mixing 4-aminoantipyrine and 2-nitro-4-(trifluoromethyl)benzoyl chloride in dried acetone to obtain a reaction mixture;
b) refluxing the reaction mixture for 30 minutes to obtain a product mixture;
c) cooling the product mixture at room temperature;
d) precipitating the cooled product mixture by pouring the product mixture into water to obtain a precipitate;
e) filtering the precipitate and washing the precipitate with 1000 ml of water;
f) drying the washed precipitate at 40° C. to obtain dried precipitate; and
g) purifying the dried precipitate to obtain solvency promotor compound of claim 9.
